(19) United States
(12) Patent Application Publication
(10) Pub. No.: US 2025/0261837 A1
(43) Pub. Date: Aug. 21, 2025

(54) SHAFTS OF MEDICAL DEVICES AND RELATED DEVICES, SYSTEMS, AND METHODS

(71) Applicant: Boston Scientific Scimed, Inc., Maple Grove, MN (US)

(72) Inventors: Joseph Phillip ALBINA, Hudson, MA (US); Greg Joseph TRUMBULL, Milford, MA (US); Evan WILDER, Malden, MA (US)

(73) Assignee: Boston Scientific Scimed, Inc., Maple Grove, MN (US)

(21) Appl. No.: 19/057,544

(22) Filed: Feb. 19, 2025

Related U.S. Application Data
(60) Provisional application No. 63/555,612, filed on Feb. 20, 2024.

(51) Int. Cl. A61B 1/012; A61B 1/00; A61B 1/005; A61B 1/018

(52) U.S. Cl. CPC A61B 1/0125; A61B 1/00071; A61B 1/0052; A61B 1/018

(57) ABSTRACT

A medical device may comprise a handle and a shaft extending distally from the handle. The shaft may include a proximal portion configured to remain external to a body lumen of a subject and a distal portion configured to be inserted into the body lumen of the subject. The proximal portion of the shaft may have a greater stiffness than the distal portion of the shaft.

### CROSS-REFERENCE TO RELATED APPLICATIONS

This application claims the benefit of priority to U.S. Provisional Application No. 63/555,612, filed on Feb. 20, 2024, which is incorporated by reference herein in its entirety.

### TECHNICAL FIELD

Various aspects of this disclosure relate generally to shafts of medical devices, and related devices, systems, and methods. In particular, aspects of the disclosure relate to shafts of medical devices having variable properties along lengths of the shafts, among other aspects.

### BACKGROUND

During an endoscopic procedure, an operator may insert a medical device into a working channel of a medical scope device, such as an endoscope or duodenoscope. The medical device inserted into the working channel may be a medical instrument or another medical scope device (e.g., a cholangioscope or another type of daughter scope). The medical device inserted into the working channel may have a shaft. A first portion of the shaft may extend through the working channel of the endoscopic scope. A second portion of the shaft may be proximal of a port of the working channel and outside of the working channel.

### SUMMARY

Each of the aspects disclosed herein may include one or more of the features described in connection with any of the other disclosed aspects. Aspects of the disclosure may relate to shafts of medical devices.

In an example, a medical device may comprise a handle and a shaft extending distally from the handle. The shaft may include a proximal portion configured to remain external to a body lumen of a subject and a distal portion configured to be inserted into a body lumen of the subject. The proximal portion of the shaft may have a greater stiffness than the distal portion of the shaft.

Any of the aspects disclosed herein may have any of the following features, alone or in any combination. The proximal portion of the shaft may have a greater outer diameter than the distal portion of the shaft. The proximal portion of the shaft may have a length of approximately 25 cm to approximately 50. cm. The proximal portion of the shaft may include one or more of a reflowed or extruded outer jacket, a metal or plastic braid, a metal or plastic coil, or a laser cut plastic or metal tubing. The distal portion of the shaft may include one or more of etched polytetrafluoroethylene, a metal or plastic braid, a reflowed or extruded jacket, or a stiffener. The medical device may include a cholangioscope. The shaft may include a tapered transition between the proximal portion of the shaft and the distal portion of the shaft.

A medical system may include the medical device. The medical system may further comprise a medical scope having a working channel. The distal portion of the shaft may be configured to be inserted into the working channel. The proximal portion of the shaft may be configured to remain external to the working channel. Any of the medical systems or devices disclosed herein may have any of the features below, alone or in any combination or subcombination. The proximal portion of the shaft may have a greater diameter than the working channel of the medical scope. The proximal portion of the shaft may have a diameter greater than approximately 4.2 mm. In a configuration in which the distal portion of the shaft is inserted into the working channel, the proximal portion of the shaft may have a loop shape. In a configuration in which the proximal portion has a loop shape, the greater stiffness of the proximal portion of the shaft may inhibit buckling of the proximal portion upon actuation of an actuator of the handle of the medical device. The actuation of the actuator may cause a steerable portion of the distal portion of the shaft to articulate. The greater stiffness of the proximal portion of the shaft may increase an articulation performance of the steerable portion. In a configuration in which the distal portion of the shaft is inserted into the working channel, a transition between the proximal portion and the distal portion may be proximal of a port of the working channel.

In another example, a medical device may comprise a handle; and a shaft extending distally from the handle. The shaft may include a proximal portion configured to remain external to a body lumen of a subject; and a distal portion configured to be inserted into a body lumen of the subject. The proximal portion of the shaft may have a greater outer diameter than the distal portion.

A medical system may include the medical device. The medical system may further comprise a medical scope having a working channel. The distal portion of the shaft may be configured to be inserted into the working channel. The proximal portion of the shaft may be configured to remain external to the working channel. The proximal portion of the shaft may have a greater diameter than the working channel of the medical scope. Any of the medical systems or devices disclosed herein may include any of the following features, alone or in any combination or subcombination. In a configuration in which the distal portion of the shaft is inserted into the working channel, the proximal portion of the shaft may have a loop shape.

In a further aspect, a medical system may comprise a first medical device, which may include a first shaft having a proximal portion configured to remain external to a body lumen of a subject; and a distal portion configured to be inserted into a body lumen of the subject. The medical system may further comprise a second medical device including a second shaft having a working channel. The distal portion of the first shaft may be configured to be inserted into the working channel. The proximal portion of the first shaft may have a greater diameter than a diameter of the working channel.

Any of the medical systems or devices disclosed herein may have any of the following features, alone or in any combination or subcombination. The first medical device may be a cholangioscope.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIGS. 1A and 1B depict proximal and distal portions, respectively, of an exemplary first medical device.

FIG. 2 depicts a medical system including the first medical device of FIGS. 1A and 1B and a second medical device.

FIG. 3A depicts another exemplary medical device.

FIGS. 3B and 3C depict aspects of a shaft of the medical device of FIG. 3A.

### DETAILED DESCRIPTION

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values+/−10% of a stated value. Unless otherwise stated, ranges disclosed herein include the end points of the ranges. The use of ordinals (e.g., first, second, third, fourth) herein is for convenient reference to different examples and does not imply any ordered or other type of relationship among identified elements. The ordinal applied to a given element is arbitrary, and any alternative ordinal may be used with the element. A period after a zero in a number indicates that the zero is significant for purposes of determining significant figures.

A first medical device, such as a cholangioscope (or other type of daughter scope) or an accessory instrument, may be inserted into a working channel of a second medical device, such as a duodenoscope or other type of scope. A distal portion of the shaft of the first medical device may extend through the working channel of the second medical device. A proximal portion of the shaft of the first medical device may be proximal (or otherwise outside) of a working channel port of the second medical device. Thus, the proximal portion of the shaft may be outside of the working channel. Thus, the proximal portion of the shaft may be external to a body lumen of a subject, whereas the distal portion of the shaft may be inserted into the body lumen of the subject. The proximal portion of the shaft may be looped to form a working loop.

In conventional devices, the proximal portion of the shaft may be so flexible as to be prone to buckling. This buckling may be detrimental to performance of the first medical device. For example, in aspects in which the first medical device is a cholangioscope or other type of daughter scope, buckling of the proximal portion of the shaft may be detrimental to articulation of the distal portion of the shaft of the first medical device. In other examples, buckling may be detrimental to actuation of other types of control wires (e.g., wires controlling end effectors). Actuation of the first medical device may compress the working loop, rather than deflecting a distal tip of the shaft of the first medical device or otherwise controlling a shaft or distal tip of the first medical device.

The disclosed aspects include a medical device with a shaft that has a reinforced proximal portion, which may improve performance (e.g., articulation performance or other actuation performance) of the medical device. The reinforced proximal portion may be stiffer than a distal portion of the shaft. In some examples, the proximal portion of the shaft (which may form a working loop) may have a diameter that is larger than a diameter of a distal portion of the shaft and/or a working channel into which the distal portion of the shaft is to be inserted.

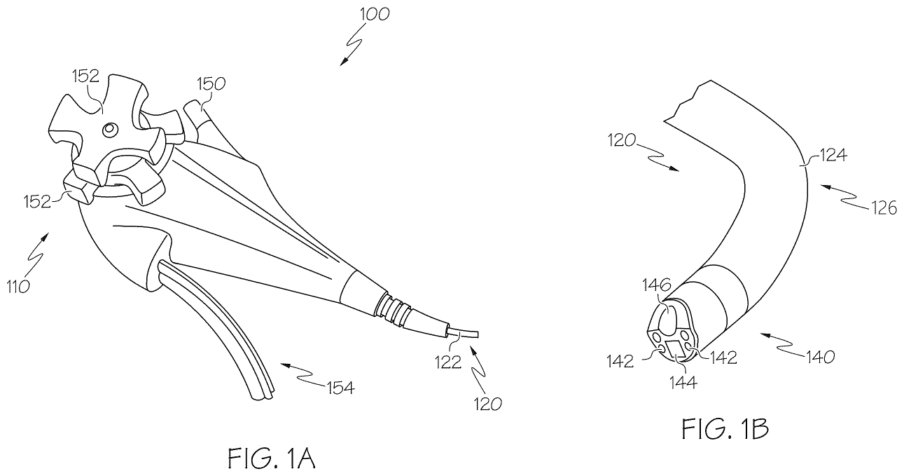

FIGS. 1A and 1B depict proximal and distal portions, respectively, of a first medical device 100. In some examples, first medical device 100 may be a cholangioscope. However, although cholangioscopes are referenced herein, it will be appreciated that first medical device 100 may be another type of daughter scope, an endoscope, a ureteroscope, a duodenoscope, a gastroscope, an endoscopic ultrasonography (“EUS”) scope, a colonoscope, a bronchoscope, a laparoscope, an arthroscope, a cystoscope, an aspiration scope, a sheath, a catheter, a tome, or any type of accessory instrument having a shaft (e.g., a needle, a snare, a basket, a stent, a knife, a stapler, etc.).

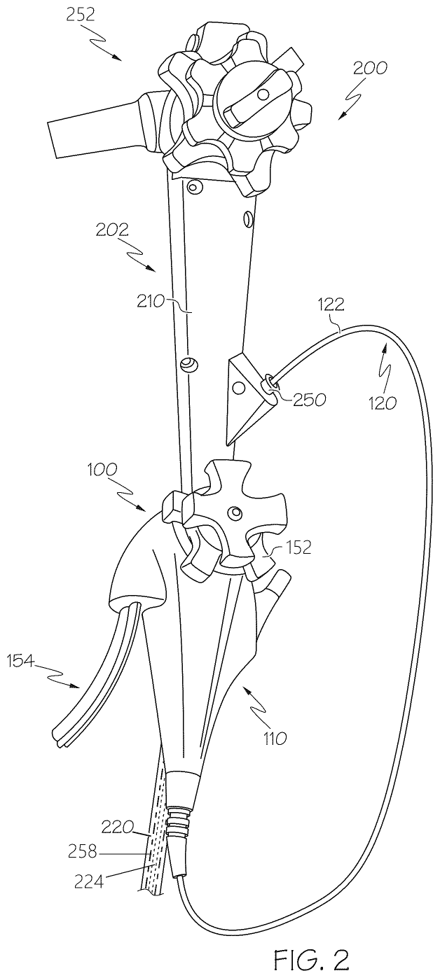

First medical device 100 may include a handle 110 and a shaft 120. Shaft 120 may have a distal tip 140 coupled to a distal end of shaft 120. Shaft 120 may include a proximal portion 122 (FIG. 1A) and a distal portion 124 (FIG. 1B). Distal portion 124 of shaft 120 and distal tip 140 may be configured to be inserted into a body lumen of a subject. For example, distal portion 124 of shaft 120 and distal tip 140 may be inserted into a working channel of a second medical device (e.g., second medical device 202, shown in FIG. 2 and described below). As described in further detail below, proximal portion 122 may remain outside of/external to the body lumen of the subject and the working channel of the second medical device. Distal portion 124 may include a steerable section 126, which may include an articulation joint or other structure (e.g., a multilumen extruded shaft or other bendable shaft) that is configured to deflect or otherwise articulate in order to steer steerable section 126 and distal tip 140.

Distal tip 140 may include one or more lighting elements 142, one or more imaging devices 144, and a distal opening 146 of a working channel of first medical device 100. One or more lighting elements 142, one or more imaging devices 144, and distal opening 146 may have any suitable arrangement. Alternatively or additionally, distal tip 140 may include one or more end effectors (e.g., graspers, snares, staplers, clips, stents, forceps, suturing devices, baskets, etc.) for performing a medical procedure at a treatment site.

Handle 110 may include a port 150, which may provide access to the working channel of first medical device 100. An accessory instrument may be inserted into port 150, advanced through the working channel, and out of distal opening 146 of distal tip 140. Handle 110 may also include one or more actuators 152 for controlling aspects of shaft 120 and/or distal tip 140. For example, one or more actuators 152 may include one or more steering knobs (or levers, sliders, etc.) for controlling articulation of steerable section 126. For example, one or more cables and/or wires may extend from one or more actuators 152 to steerable section 126. Tensioning or de-tensioning of the cables and/or wires may cause steerable section 126 to deflect in one or more of an up/down or left/right direction. In alternatives, actuator(s) 152 may be used to control an end effector of distal tip 140 using one or more control wires or cables. Actuator(s) 152 may additionally or alternatively control electronic elements of distal tip 140 or deliver air, suction, and/or water.

An umbilicus 154 may extend from handle 110. Umbilicus 154 may include one or more cables, tubes, or other conduits for conveying electrical signals, air, water, and/or suction to or from handle 110, shaft 120, and distal tip 140. Umbilicus 154 may include one or more proximal connectors (not shown) for connecting to capital equipment, which may supply electrical controls, air, water, and/or suction.

FIG. 2 depicts a medical system 200, including first medical device 100 and a second medical device 202. Second medical device 202 may be, for example, a duodenoscope or other type of medical scope. Although a duodenoscope is referenced herein, it will be appreciated that second medical device 202 may be any medical device having a working channel extending from a proximal end to a distal end, such as a ureteroscope, an endoscope, a gastroscope, an endoscopic ultrasonography (“EUS”) scope, a colonoscope, a bronchoscope, a laparoscope, an arthroscope, a cystoscope, an aspiration scope, a sheath, or a catheter.

Second medical device 202 may include a handle 210 and a shaft 220. Although not shown in FIG. 2, a distal tip having any of the features of distal tip 140 may be at a distal end of shaft 220. Shaft 220 may be insertable into a body lumen of a subject in order to perform a procedure at a target site in the body lumen or other portion of the body.

Handle 210 may include a port 250. Port 250 may provide access to a working channel 258 of second medical device 202, including shaft 220. A medical device (e.g., first medical device 100) may be configured to be inserted into port 250, advanced through working channel 258 of shaft 220, and extended distally from the distal tip of second medical device 202. Working channel 258 is shown in dotted-lines within shaft 220 to illustrate working channel 258 extending through an interior portion of shaft 220. Handle 210 may also include one or more actuators 252 for controlling aspects of shaft 220 and/or a distal tip of second medical device 202. For example, actuators 252 may include one or more knobs, levers, sliders, joysticks, buttons, or the like for articulating a distal portion of shaft 220. One or more actuators 252 further include levers, knobs, buttons, sliders, buttons, or the like for locking the distal portion of shaft 220 from articulating. One or more actuators 252 may also include one or more actuators for capturing images from an imaging device of a distal tip of second medical device 202, for delivering air, water, and/or suction to a distal tip of second medical device 202.

As shown in FIG. 2, handle 110 of first medical device 100 may be coupled to handle 210 of second medical device 202. For example, a strap, clamp, or the like may couple handle 110 of first medical device 100 to handle 210 of second medical device 202. Distal tip 140 may be inserted into port 250, and distal portion 124 of shaft 120 may be advanced through shaft 220 until distal tip 140 extends from a distal tip of second medical device 202. Because shaft 220 may be inserted into a body lumen of a subject, distal portion 124 of shaft 120 may also be inserted into the body lumen of the subject. In examples, a total length of shaft 120 may be approximately 200. cm long, and distal portion 124 may be approximately 150. cm to approximately 175 cm long.

As shown in FIG. 2, in a configuration in which distal portion 124 has been inserted into working channel 258 of second medical device 202, proximal portion 122 of shaft 120 of first medical device 100 may remain proximal of and outside of port 250 and outside of working channel 258 of second medical device 202. Thus, proximal portion 122 may remain external to a body (and a body lumen) of a subject. Proximal portion 122 may form a loop shape (e.g., a working loop). Handle 110 may be coupled to handle 210 distally of port 250, such that proximal portion 122 loops proximally before entering port 250. For example, proximal portion 122 may extend distally from a distal end of handle 110 and may then be looped back in a proximal direction toward port 250, before then extending distally into port 250. In examples, a length of proximal portion 122 may be approximately 25 cm to approximately 50. cm.

Distal portion 124 may be supported by working channel 258, which may constrain distal portion 124. Such constraints of working channel 258 on distal portion 124 may improve a transmission of articulation controls from one or more actuators 152 to steerable section 126. In other words, support of working channel 258 on distal portion 124 may help the steering wires and/or cables extending from one or more actuators 152 to steerable section 126 to appropriate shorten or lengthen (move proximally or distally relative to distal portion 124) within distal portion 124, so that steering control is transmitted to steerable section 126.

In contrast, conventional proximal portions of shaft 120 are outside of and are not supported by working channel 258. Upon actuation of one or more actuators 152, the proximal, looped portion may buckle or bend due to action (e.g., movement or tensioning/detensioning) of the steering wires/cables. This buckling of conventional proximal portions may inhibit the steering wires or cables from transmitting tension to steerable section 126 in order to deflect steerable section 126. In other words, actuation of one or more actuators 152 may cause compression of conventional proximal portions (working loops), rather than deflection of steerable section 126.

To address these issues with conventional scopes, proximal portion 122 and distal portion 124 of shaft 120 may have different properties from one another. For example, proximal portion 122 may be stiffer than distal portion 124. For example, proximal portion 122 may be reinforced with respect to distal portion 124. An increased stiffness of proximal portion 122 as compared with distal portion 124 may help to transmit steering/articulation forces from handle 110 to distal tip 140. For example, an increased stiffness of proximal portion 122 may help to inhibit it from buckling or compressing.

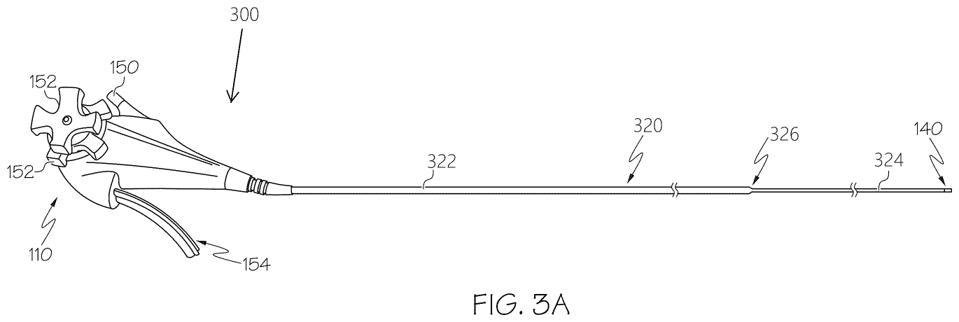

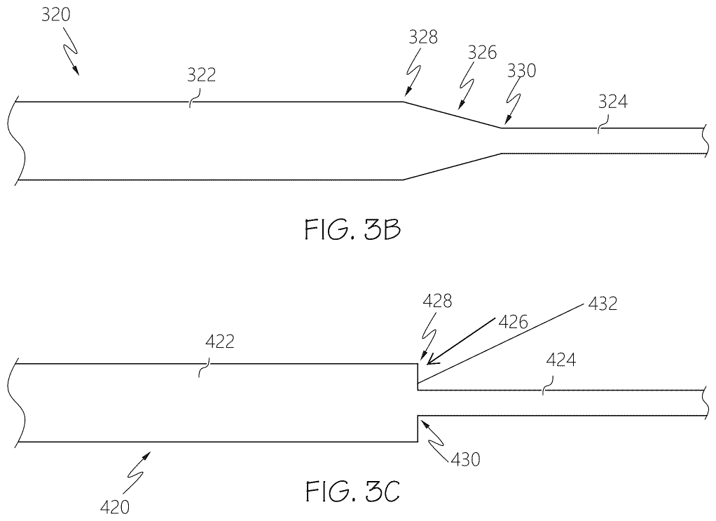

FIGS. 3A-3B show an alternative exemplary first medical device 300 that may have reinforcement to prevent or inhibit buckling of a working loop of first medical device 300. Unless otherwise specified herein, first medical device 300 may have any of the properties of first medical device 100. First medical device 300 may have a shaft 320, having any of the properties of shaft 120, unless otherwise specified herein. Shaft 320 may have a proximal portion 322 (having any of the properties of proximal portion 122) and a distal portion 324 (having any of the properties of distal portion 124). Proximal portion 322 may be configured to remain external to working channel 258 of second medical device 202 and external to a body lumen of a subject. Distal portion 324 may be configured to be inserted into and extend through working channel 258 and, thus, be inserted into a body lumen of a subject.

Distal portion 324 may have an outer diameter such that distal portion 324 may be inserted into port 250 and extended through working channel 258 of shaft 220. In other words, an outer diameter of distal portion 324 may be smaller than an inner diameter of working channel 258. For example, working channel 258 may have an inner diameter of approximately 3.0 mm to approximately 5.0 mm, approximately 3.8 mm to approximately 4.4 mm, approximately 4.2 mm, or any other suitable diameter. Distal portion 324 may have an outer diameter of approximately 3.0 mm to approximately 4.2 mm, approximately 3.2 mm to approximately 4.0 mm, approximately 3.6 mm, or any other suitable diameter.

In contrast, proximal portion 322 may have an outer diameter that is larger than an outer diameter of distal portion 324 and/or larger than an inner diameter of working channel 258 because proximal portion 322 may not be insertable into working channel 258. For example, proximal portion 322 may have an outer diameter that is greater than approximately 3.6 mm (which may correspond to an outer diameter of distal portion 124) or greater than approximately 4.2 mm (which may correspond to an inner diameter of working channel 256). Proximal portion 322 may be reinforced to stiffen all or a portion of proximal portion 322 as compared to distal portion 324.

Distal portion 324 of shaft 320 may include, for example, layers of etched polytetrafluoroethylene (PTFE), braid (e.g., braided metal or plastic), and a jacket (e.g., a reflowed or extruded jacket). Additionally or alternatively, distal portion 324 may include layers of PTFE and a stiffener. By contrast, proximal portion 322 of shaft 320 may include a composite shaft with one or more of a reflowed or extruded outer jacket, a metal or plastic braid, a metal or plastic coil, and/or a laser cut plastic or metal tubing. For example, proximal portion 322 may include fluorinated ethylene propylene (FEP), such as an FEP heat-shrink. Proximal portion 322 may include additional or thicker layers of material, as compared with distal portion 324. As compared with distal portion 324, proximal portion 322 may be stiffer and may have a larger outer diameter.

In some examples, inner layers/materials of proximal portion 322 may be the same as the layers/materials of distal portion 324. Proximal portion 322 may have additional outer layers (such as the layers discussed above) added as outer layers of proximal portion 322. In other words, layers of distal portion 324 may extend proximally into proximal portion 322, and proximal portion 322 may further have additional outer layers added on an outer surface of proximal portion 322. Alternatively, some or all of the materials/layers of distal portion 324 may be omitted from proximal portion 322, and proximal portion 322 may have different materials and/or layers from distal portion 324.

Shaft 320, with distal portion 324 and proximal portion 322 having different properties, may offer advantages as compared to a shaft with uniform properties, such as a shaft that increases a stiffness of the entire shaft in order to create a stiffer working loop. For example, stiffening an entire length of a shaft may make it more difficult to advance a distal portion of the shaft along working channel 258 of second medical device 202 or to deflect the distal portion of the shaft with an elevator at the distal tip of second medical device 202.

As shown in FIGS. 3A and 3B, a transition 326 between proximal portion 322 and distal portion 324 may be tapered. Transition 326 may taper inward in a distal direction, from a widest portion 328 of transition 326 to a narrowest portion 330 of transition 326. Widest portion 328 of transition 326 may be proximal of narrowest portion 330 of transition 326. Widest portion 328 of transition 326 may have a same width/diameter as proximal portion 322 of shaft 320. Narrowest portion 330 of transition 326 may have a same width/diameter as distal portion 324 of shaft 320. In examples, an outer surface of shaft 320 may be molded, re-flowed, extruded, or otherwise formed to create transition 326. In addition or alternatively, additional layers may progressively be added to distal portion 324 moving in a proximal direction, to create a tapered shape of transition 326. Tapered transition 326 may have any suitable angle or shape. When device 300 is inserted into second medical device 202, transition 326 may be proximal of port 250.

FIG. 3C shows a portion of an alternative shaft 420 that may be used in place of shaft 320 with device 300. Shaft 420 may have any of the properties of shafts 120, 320, unless otherwise specified. Whereas transition 326 may be tapered, a transition 426 of shaft 420 may be a sharp or discrete transition. A widest portion 428 of transition 426 may have a same diameter/width as a proximal portion 422 of shaft 420. A narrowest portion 430 of transition 426 may have a same diameter/width as distal portion 424 of shaft 420. For example, a widest portion 428 of transition 426 may be axially aligned with a narrowest portion 430 of transition 426. Thus, transition 426 may extend perpendicularly to a central longitudinal axis of shaft 420. Transition 426 may form a shelf shape, and may include a distal facing surface 432 extending around a distalmost end of distal portion 424.

In one example, distal portion 424 and proximal portion 422 may have different compositions and may be coupled together at transition 426. Alternatively, a core of proximal portion 422 may have the same layers as distal portion 424, and additional layers may be added to proximal portion 422 in order to create a larger thickness and greater stiffness of proximal portion 422.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical device comprising:
a handle; and
a shaft extending distally from the handle, wherein the shaft includes:
a proximal portion configured to remain external to a body lumen of a subject; and
a distal portion configured to be inserted into the body lumen of the subject;
wherein the proximal portion of the shaft has a greater stiffness than the distal portion of the shaft.

2. The medical device of claim 1, wherein the proximal portion of the shaft has a greater outer diameter than the distal portion of the shaft.

3. The medical device of claim 1, wherein the proximal portion of the shaft has a length of approximately 25 cm to approximately 50. cm.

4. The medical device of claim 1, wherein the proximal portion of the shaft includes one or more of a reflowed or extruded outer jacket, a metal or plastic braid, a metal or plastic coil, or a laser cut plastic or metal tubing.

5. The medical device of claim 4, wherein the distal portion of the shaft includes one or more of etched polytetrafluoroethylene, a metal or plastic braid, a reflowed or extruded jacket, or a stiffener.

6. The medical device of claim 1, wherein the medical device includes a cholangioscope.

7. The medical device of claim 1, wherein the shaft includes a tapered transition between the proximal portion of the shaft and the distal portion of the shaft.

8. A medical system including the medical device of claim 1, the medical system further comprising a medical scope having a working channel, wherein the distal portion of the shaft is configured to be inserted into the working channel, and wherein the proximal portion of the shaft is configured to remain external to the working channel.

9. The medical system of claim 8, wherein the proximal portion of the shaft has a greater diameter than the working channel of the medical scope.

10. The medical system of claim 8, wherein the proximal portion of the shaft has a diameter greater than approximately 4.2 mm.

11. The medical system of claim 8, wherein, in a configuration in which the distal portion of the shaft is inserted into the working channel, the proximal portion of the shaft has a loop shape.

12. The medical system of claim 11, wherein, in a configuration in which the proximal portion has a loop shape, the greater stiffness of the proximal portion of the shaft inhibits buckling of the proximal portion upon actuation of an actuator of the handle of the medical device.

13. The medical system of claim 12, wherein the actuation of the actuator causes a steerable portion of the distal portion of the shaft to articulate.

14. The medical system of claim 13, wherein the greater stiffness of the proximal portion of the shaft increases an articulation performance of the steerable portion.

15. The medical system of claim 8, wherein, in a configuration in which the distal portion of the shaft is inserted into the working channel, a transition between the proximal portion and the distal portion is proximal of a port of the working channel.

16. A medical device comprising:
a handle; and
a shaft extending distally from the handle, wherein the shaft includes:
a proximal portion configured to remain external to a body lumen of a subject; and
a distal portion configured to be inserted into the body lumen of the subject;
wherein the proximal portion of the shaft has a greater outer diameter than the distal portion.

17. A medical system including the medical device of claim 16, the medical system further comprising a medical scope having a working channel, wherein the distal portion of the shaft is configured to be inserted into the working channel, wherein the proximal portion of the shaft is configured to remain external to the working channel, and wherein the proximal portion of the shaft has a greater diameter than the working channel of the medical scope.

18. The medical system of claim 17, wherein, in a configuration in which the distal portion of the shaft is inserted into the working channel, the proximal portion of the shaft has a loop shape.

19. A medical system comprising:
a first medical device including:
a first shaft including:
a proximal portion configured to remain external to a body lumen of a subject; and
a distal portion configured to be inserted into the body lumen of the subject; and
a second medical device including a second shaft having a working channel;
wherein the distal portion of the first shaft is configured to be inserted into the working channel, and wherein the proximal portion of the first shaft has a greater diameter than a diameter of the working channel.

20. The medical system of claim 19, wherein the first medical device is a cholangioscope.
